(19) United States
(12) Plant Patent Application Publication
(10) Pub. No.: US 2006/0195955 P1
(43) Pub. Date: Aug. 31, 2006

(54) Rose named 'Her Ice'

(76) Inventor: Eunice Emmerantia Herholdt, Gauteng (ZA)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/920,792

(22) Filed: Nov. 22, 2004

(51) Int. Cl. A01H 5/00

(52) U.S. Cl. PLT/218

(57) ABSTRACT

This invention relates to a new and distinct variety of rose plant of the floribunda class, substantially as herein shown and described, characterized particularly as to the novelty by its firm, double, centered, white flower.

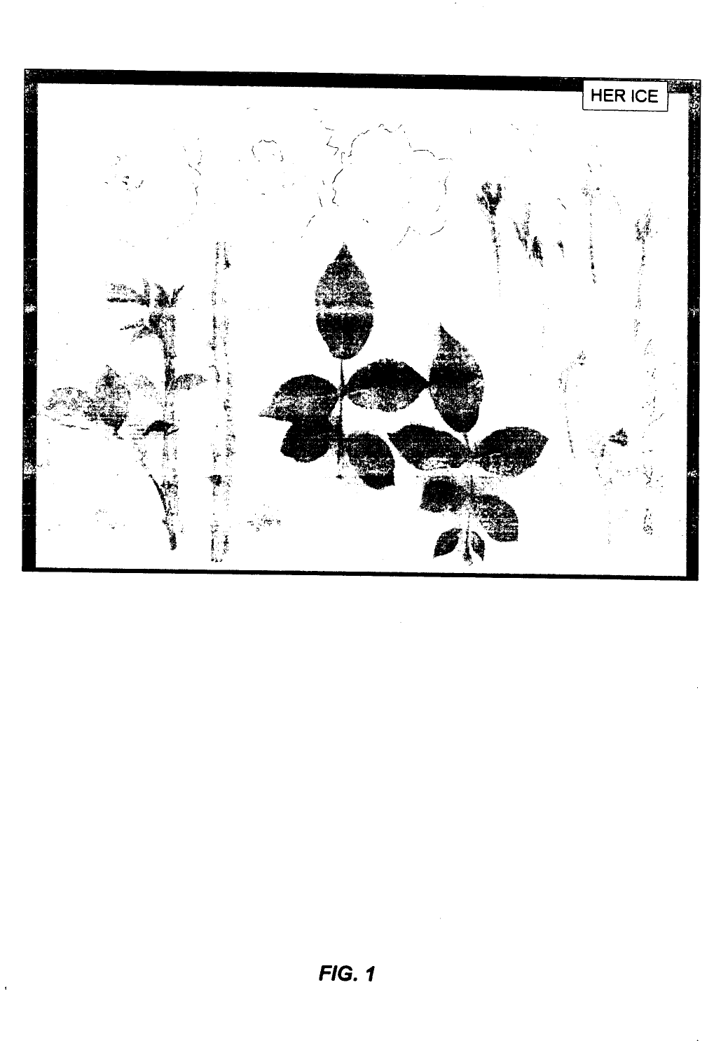

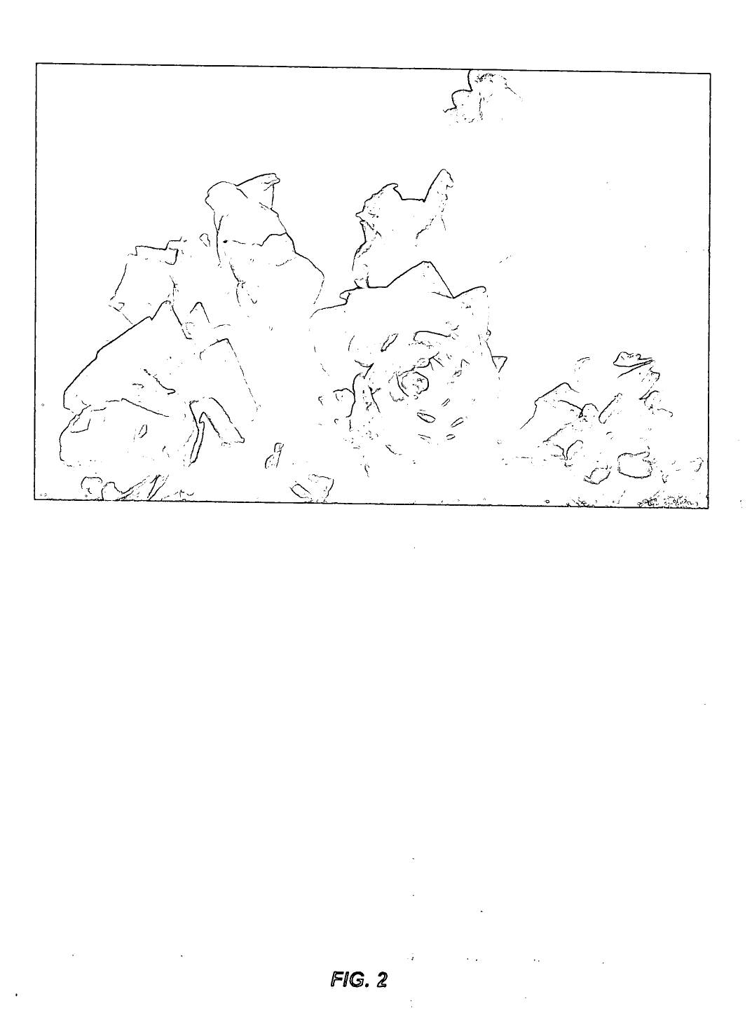

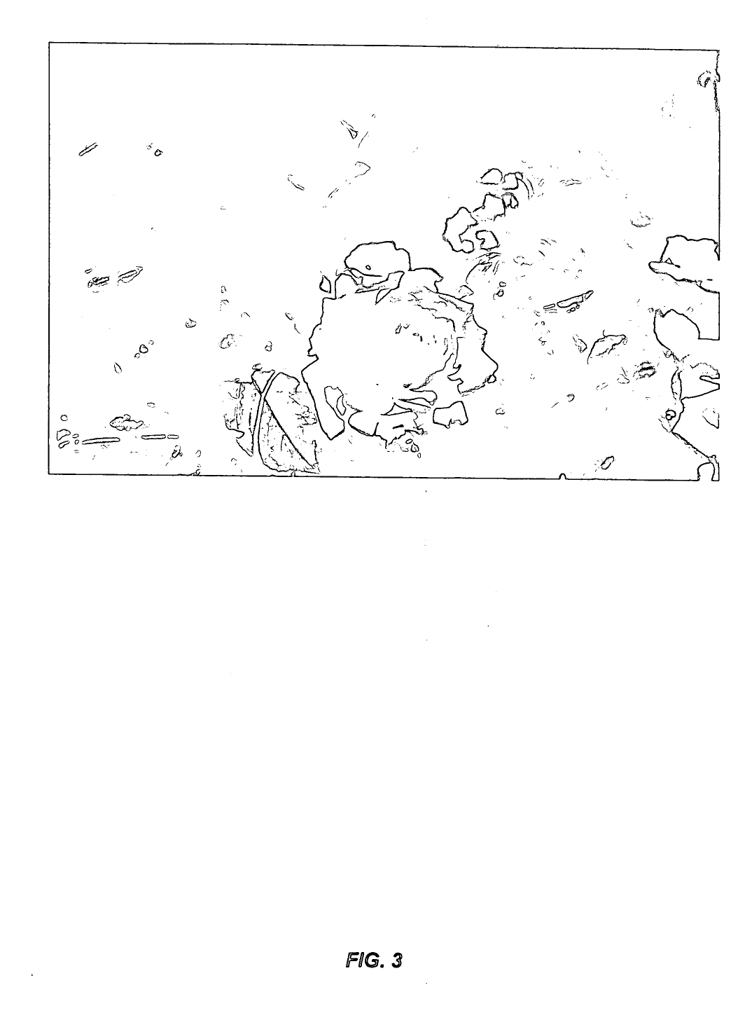

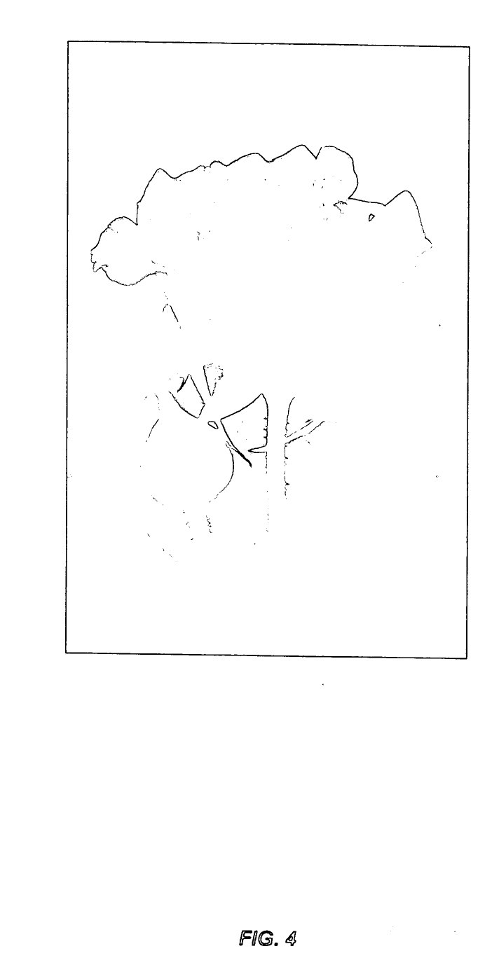

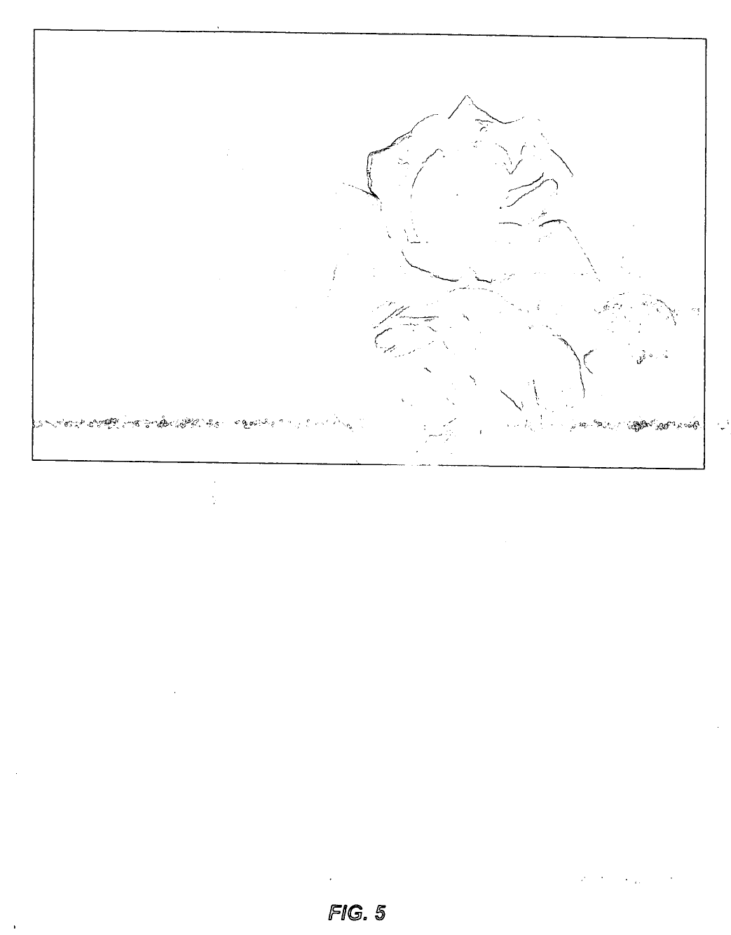

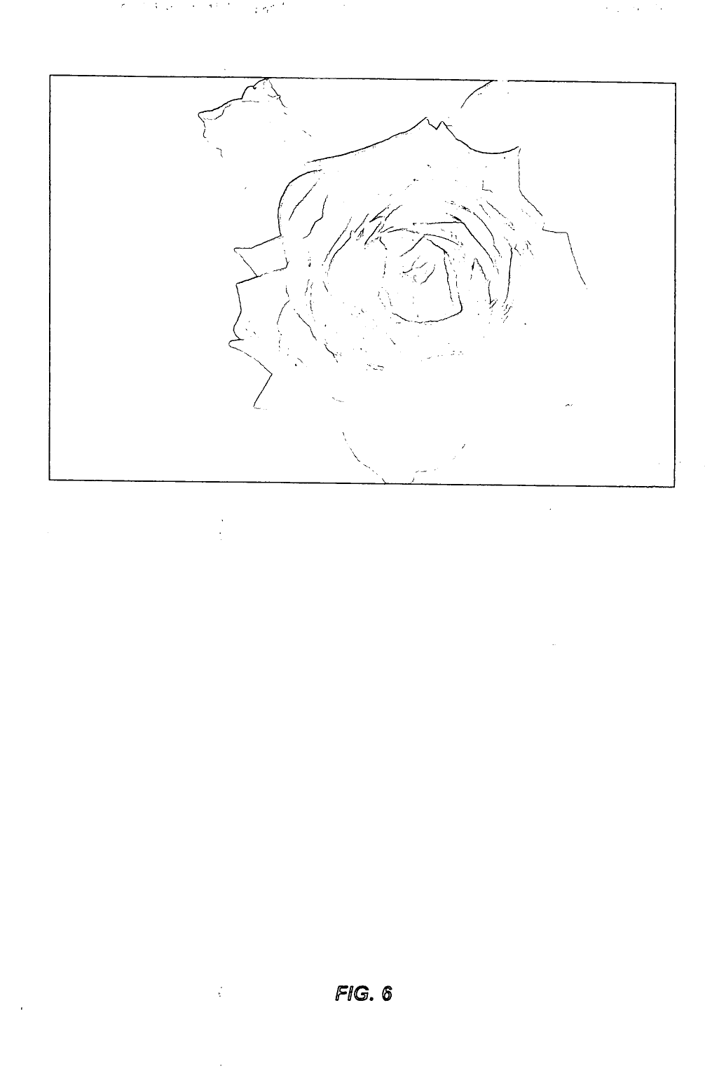

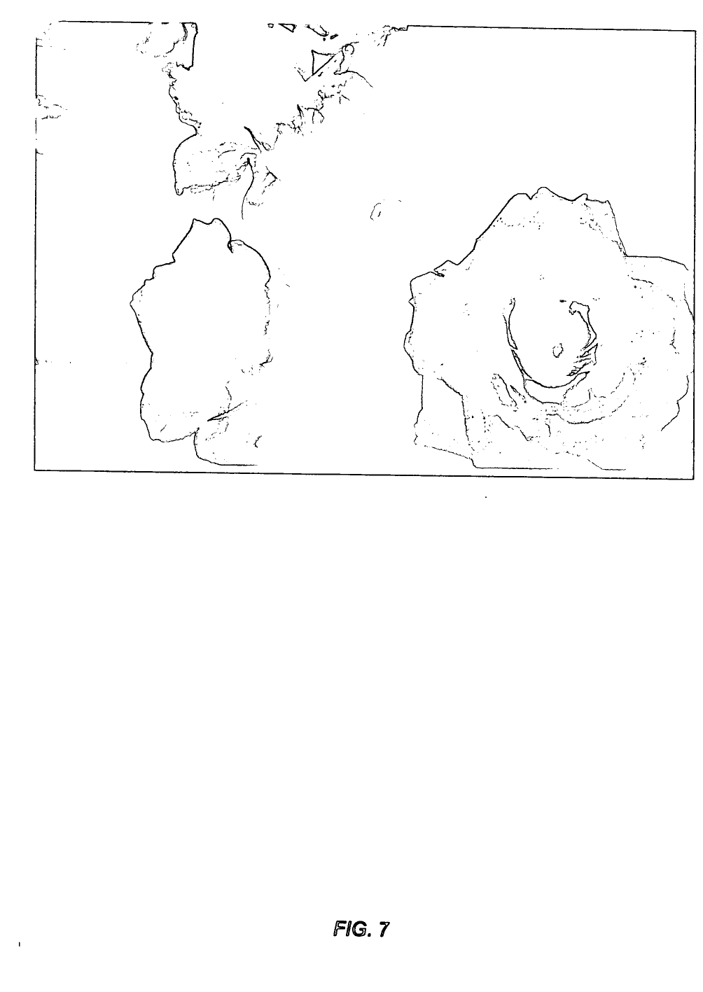

### GENUS AND SPECIES

The rose variety of this invention is botanically identified as Rosa hybrida.

### VARIETY DENOMINATION

The variety denomination is ‘Her Ice.’

### BACKGROUND OF THE INVENTION

The present invention relates to a new and distinct variety of rose plant of the floribunda class which was originated from a seed of the variety ‘Iceberg’. ‘Iceberg” is not patented to Applicant's knowledge. The pollen parent is unknown.

### BRIEF SUMMARY OF THE INVENTION

The new rose variety has a vigorous, medium bushy plant habit, good disease resistance and ability to grow well on its own roots, combined with profuse flowering, many petaled flowers and glossy foliage. The following unique combination of characteristics are outstanding in the new variety and distinguish it from its parent, as well as from all other varieties of which the Applicant is aware:

1. Firm, large double flower, which is centered;

2. Slightly scented white flower;

3. Constant blooming, flowers last longer than Iceberg (at least 7 days on the plant and longer than 7 days as a cut flower);

4. Medium, vigorous, narrow bushy, well-branched plant;

5. Good disease resistance; and

6. Ability to grow well on its own roots.

### DETAILED DESCRIPTION OF THE INVENTION

Asexual reproduction of this new variety by budding and softwood cuttings shows that the foregoing and all other characteristics and distinctions come true to form and are established and transmitted through succeeding propagations.

The following is a detailed description of new rose cultivar ‘HEr Ice’ with color descriptions using terminology in accordance with The Royal Horticultural Society (London) 2^{ND} edition color chart, except where ordinary dictionary significance of color is indicated.
Parentage:
   Seed parent.—‘Iceberg’ (not patented).
Classification:
   Botanical.—Rosa hybrida.
   Commercial —Floribunda.

### Flower

Observations made from specimens grown in a nursery environment at Eikenhof, Johannesburg, South Africa from October 2003 to July 2004.
Blooming habit: Continuous.
Bud:
   Size.—1⅜ inches (3 cm) long when the petals start to unfurl.
   Form.—The bud form is ovate.
   Color.—When sepals first divide, bud color is White Group 155D.
   Sepals.—Color: Yellow-Green Group 144A. Surface texture: Smooth. There are five sepals.
   Oveary.—outside — Color: Light Green Group 146C. Shape: Funnel. Size: Medium. Surface: Smooth.
   Peduncle.—Length: Medium 2⅕ inches (6 cm). Surface: Medium prickles. Color: Yellow-Green Group 146D. Strength: Good.
Bloom:
   Size.—Medium. Average open size is 2⅗ inches (6.7 cm).
   Borne.—Several together in clusters of five to nine blooms.
   Stems.—Strength: Strong. Average length is about 8 inches (20 cm).
   Form.—Bloom is centred. Permanence: Stays centred and firm (does not flatten).
   Petalage.—Number of petals under normal conditions: 50-70, on average 68.4.
   Color.—The upper and reverse sides of the entire petals are White Group 155D.
   Variegations.—None.
   Discoloration.—None.
   Fragrance.—Slight to medium.
Petal:
   Texture.—Medium.
   Shape.—Round to deltoid.
   Form.—Tips slightly recurved.
   Average Length.—1⅖ inches (3.5 cm).
   Average Width.—1 inch (2.6 cm).
   Arrangement.—Imbricated. Side view of upper and lower parts of bloom flat. Top view of bloom is irregular and rounded. Arrangement is similar to old English Rose. Petaloids in the centre are abundant, regular in shape and ¼-⅛ inches long. Outer centre petaloids are ¾ inches long.
   Persistance.—Petals drop off cleanly before drying.
   Lastingness.—On the plant: Good (at least seven days). As a cut flower: Good (over seven days).
Reproductive parts:
   Anthers.—Size: Medium. Quantity: Many. Color: Yellow-Orange Group 22B. Arrangement: Regular around styles.
   Filaments.—Color: Yellow Group 2C.
   Pollen.—Present.
   Styles.—Color: Yellow-Green Group 154D.
   Stigmas.—Color: White Group 154D.

### Plant

Form.—Bushy narrow.
Growth.—Vigorous; bushy; branching. At maturity, height is 36 inches (92 cm), width is 27 inches (69 cm).
Foliage.—Number of leaflets on normal mid-stem leaves: Seven.
Size.—Small to Medium (2 inches long times 1 inch wide).
Quantity.—Abundant.
Color.—New foliage: Upper and lower surfaces: Green-Red. Old foliage: Upper surface: Green Group 146A. Lower surface: Green Group 146B.
Leaflets:
   Shape.—Obtuse.
   Texture.—Leathery; glossy.
   Edge.—Serrated.
   Serration.—Single; small.
   Petiole rachis.—Color: Upper: Yellow-Green Group 144A. Lower: Green Group 145A.
   Petiole underside.—Smooth.
   Stipules.—Medium (about ¾ inch); serrated.
   Disease resistance.—Resistant to mildew and rust; susceptible to blackspot under normal growing conditions.
Wood:
   New wood.—Color: Green Group 146B. Bark: Smooth.
   Old wood.—Color: Green. Bark: Smooth.
Prickles:
   Quantity.—On main canes from base: Ordinary. On laterals from main canes: Ordinary.
   Form.—Medium.
   Color when young.—White/Beige.
   Color when mature.—Light Brown Group 173D.
Small prickles:
   Quantity.—On main stalks: None. On laterals: None.

What is claimed is:

1. A new and distinct rose variety having the characteristics substantially as described and illustrated herein.
